(19) United States
(12) Patent Application Publication
(10) Pub. No.: US 2024/0391157 A1
(43) Pub. Date: Nov. 28, 2024

(54) Methods and Apparatus for Folding Ultrasound Transducer Assemblies

(71) Applicant: Veran Medical Technologies, Inc., St. Louis, MO (US)

(72) Inventors: Christopher Lee, St. Louis, MO (US); Abigail Filsinger, St. Louis, MO (US)

(73) Assignee: Veran Medical Technologies, Inc., St. Louis, MO (US)

(21) Appl. No.: 18/321,111

(22) Filed: May 22, 2023

(51) Int. Cl. A61B 8/00; A61B 8/12

(52) U.S. Cl. CPC B29C 53/46; B29C 65/48

(57) ABSTRACT

An apparatus and its method of use in folding a ultrasound transducer assembly, is disclosed. The apparatus includes a base which define two pivot channels therethrough, of which each support an axle. A folding platform has a first portion pivotally disposed about one axle and the second portion pivotally disposed about the other axle. The folding platform is moveable between an open position and a closed position. In the open position an interior faces of the first portion and second portion are in substantially parallel alignment with a top surface of the base. In the closed position the interior faces of each portion are substantially perpendicular to the top surface of the base and interior faces define a folding channel. A ultrasound transducer assembly positioned on the folding platform during actuation from the open position to the closed position is folded into a substantially taco-like shape within the folding channel.

### TECHNICAL FIELD

Embodiments disclosed herein are directed to tools and techniques for the preparation of medical devices used in detecting, sampling staging and/or treating target tissues within the anatomy of a patient, such as the lungs. Some embodiments are more specifically directed to a tool or tooling utilized for folding of a transducer assembly such as of the type utilized in an endobronchial ultrasound (EBUS) catheter or device.

### BACKGROUND

A solitary pulmonary nodule (SPN) is an isolated mass in the lung that is smaller than three centimeters in diameter and is surrounded by normal tissue. SPNs can be identified through common medical imaging techniques such as computerized tomography (CT) scans and positron emission tomography (PET) scans. In most cases SPNs and other lung nodules are merely benign tumors. In other cases, these SPNs are malignant cancers which must be treated to prevent premature mortality.

Diagnosis of identified SPNs cannot be performed via medical imaging alone but instead requires a biopsy which may be performed via an EBUS device equipped with real-time sampling functionality. Real-time sampling with an EBUS device typically includes navigating the EBUS device through a patient's airways to a target nodule that was previously identified in a medical image so that the target nodule is visible in a real-time ultrasound (US) image. Then, while the target nodule is viewed in the US image, a sampling needle is extended from a working channel of the EBUS device into the target nodule to obtain a sample thereof.

Existing EBUS devices are too large to reach deep into the secondary airway branches of the lungs in part because of the difficulties involved with manufacturing miniaturized US transducers with a high degree of quality and repeatability. SPNs within the secondary airway branches which cannot be reached for sampling using existing EBUS devices may be reached percutaneously, but percutaneous sampling is generally unpreferred compared to endobronchial sampling.

Accordingly, there is a clinical need for further miniaturization of US transducers suitable for use in EBUS devices. One technique for reducing the width or profile of a transducer assembly is to fold the assembly essentially in half so that it resembles a “taco” in shape. Folding the transducer assembly in such a manner nearly halves the area taken up by the US transducer assembly while only slightly increasing its depth (thickness). To provide such a folded transducer assembly requires that the transducer assembly be comprised of a flexible substrate on which a US transducer and associated components are mounted, as well as a tool and technique for folding the transducer assembly into the desired taco-like configuration.

Aspects of a flexible US transducer assembly are shown and described in U.S. application Ser. No. 17/970,696, filed on Oct. 21, 2022 and entitled Low Profile Ultrasound Catheter and System, the entire content of which is incorporated herein by reference.

Embodiments of a tool for folding such a US transducer assembly and techniques utilized during the folding procedure, as well as related aspects for ensuring that the transducer assembly retain its reduced profile configuration following folding, are shown in the attached drawings and expounded upon below.

### SUMMARY

An apparatus or tool is disclosed herein which is capable of receiving a US transducer assembly into a folding platform, and when the tool is actuated, will fold the US transducer assembly into a taco shape/configuration without detrimental impact to the components of the US transducer assembly, while ensuring that the assembly maintains a configuration capable of performing its intended functions.

The tool is comprised of a base upon which a folding platform (for folding a US transducer assembly) is pivotally mounted. The folding platform is comprised of two spaced apart portions of which each portion is independently pivotable relative to the base. Each portion defines a half of the folding platform.

In some embodiments, each portion of the folding platform includes a wing or arm to allow for manual actuation of the portions. Each portion of the folding platform and its associated arm are pivotable between a first or open position and a second or closed position. The folding platform is configured such that when the portions are in the first position the folding platform is in an open configuration that accommodates, and is capable of receiving, a US transducer assembly in its manufactured or flattened out state. When then the portions of the folding platform are pivoted to their second position, the folding platform transitions from the open position to a closed or folded position, which forces a US transducer assembly positioned on the folding platform to take on a folded, taco-like shape. In the closed position, the portions of folding platform define a folding channel.

In some embodiments, one or both portions of the folding platform and/or the arms are provided with stops or protrusions, which are sized and positioned to ensure that when the folding platform is in the closed position, the width of the folding channel is precisely established to ensure that a US transducer assembly contained therein is folded to a desired degree and configuration.

In some embodiments, each portion of the folding platform is equipped with at least one retaining mechanism configured to hold the folding platform in the closed position. In at least one embodiment, each arm includes a magnet. The magnets in one arm are in opposite polarity to the magnets in the other arm, such that when the arms are in the second position, the opposing magnets will retain the arms in the second position until they are manually pulled apart.

In at least one embodiment, the folding platform, in the closed position, defines one or more adhesive injection openings, which are in communication with the folding channel. The adhesive injection opening(s) are positioned such that when a US transducer assembly is positioned within the folding channel and adhesive is injected into the folding channel, the adhesive will be directed into the interior or fold of the taco-like shaped (folded) US transducer assembly. The adhesive, once set or cured, maintains the folded taco-like shape of the US transducer assembly once it is removed from the tool following the folding procedure.

These and other elements and features of the disclosed apparatus and method are presented in greater detail in the following description and accompanying illustrations.

### BRIEF DESCRIPTION OF THE DRAWINGS

FIG. 1 is a front perspective view of an embodiment of a folding apparatus or tool having a folding platform and arms shown in the open position.

FIG. 2 is a top-down view of a US transducer assembly, which includes a testing pad, shown in the flattened or manufactured state.

FIG. 3 is a front perspective view of the tool shown in FIG. 1, with the US transducer assembly of FIG. 2 shown placed on the folding platform of the tool.

FIG. 4 is a front perspective view of the tool shown in FIG. 3, wherein the folding platform and arms have been placed in the closed position.

FIG. 5 is a rear perspective view of the tool shown in FIG. 4 FIG. 6 is a front side-on view of the tool shown in FIG. 4.

FIG. 7 is a front perspective view of the US transducer assembly shown in the folded state (folded as a result of the tool folding the assembly in the manner shown in FIGS. 4-6).

FIG. 8 is a partial, detailed, top perspective view of the tool shown in FIGS. 4-6 illustrating the position of adhesive injection openings in communication with the folding channel formed by the folding platform being in the closed position.

FIG. 9 is a side on view of the US transducer assembly of FIG. 7 depicted with adhesive having been injected into the fold or interior of the assembly in the folded state.

### DETAILED DESCRIPTION

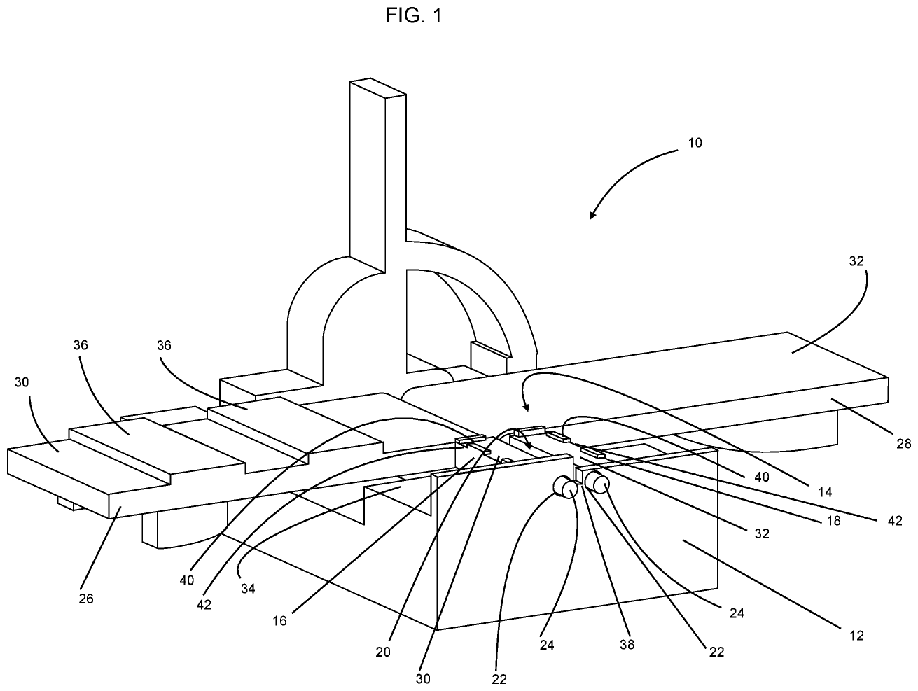

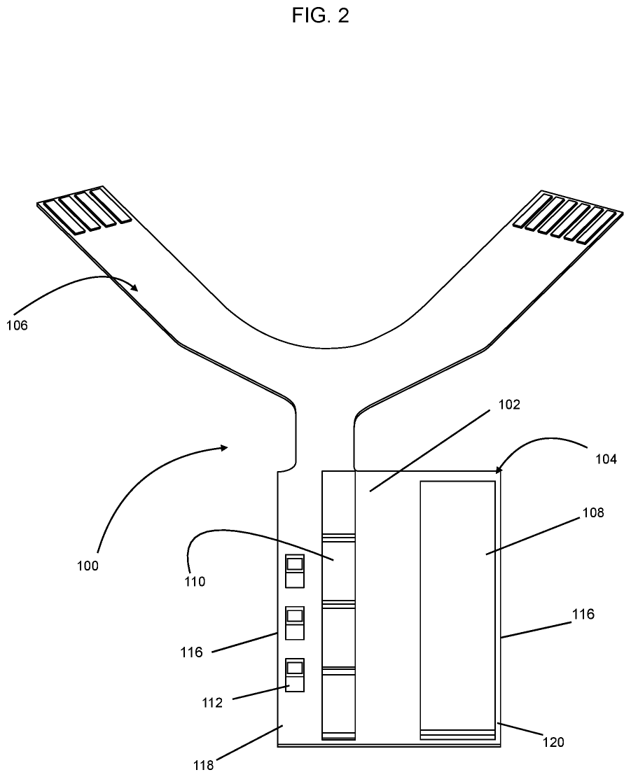

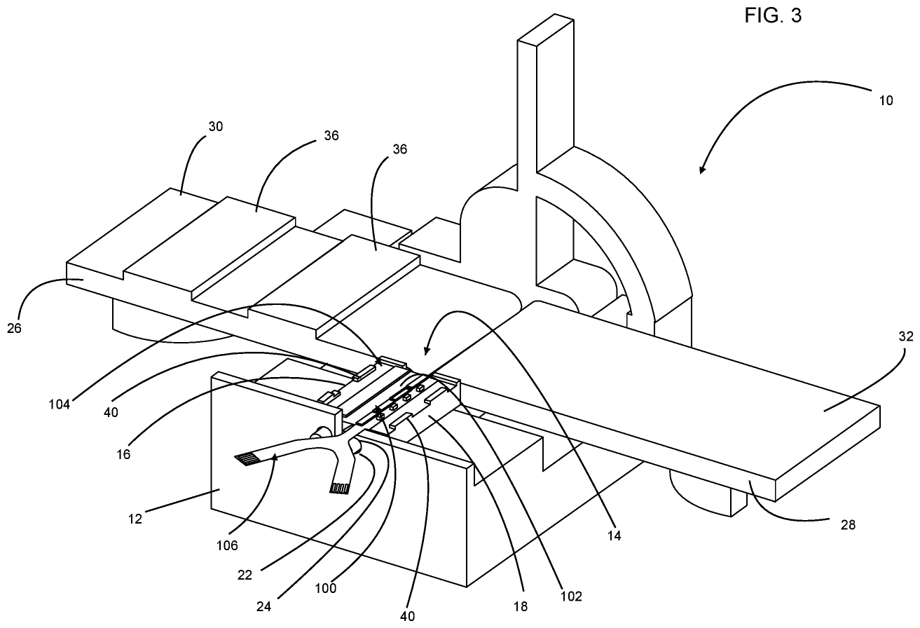

As mentioned above, embodiments of the disclosure are directed to an apparatus or tool for folding a US transducer assembly in order to reduce its effective width or profile to effectively half its nominal or manufactured state. Such a reduction in profile allows the assembly to be utilized as a component of an EBUS device capable of insertion into the relatively narrow secondary airway passages of the lungs, which conventional EBUS devices are unable to transit. An example of such a tool 10 is shown in FIG. 1. An example of the type of U.S. transducer assembly 100 that the tool 10 is configured to fold is depicted in FIG. 2, wherein the assembly 100 is shown in its manufactured or pre-folded state.

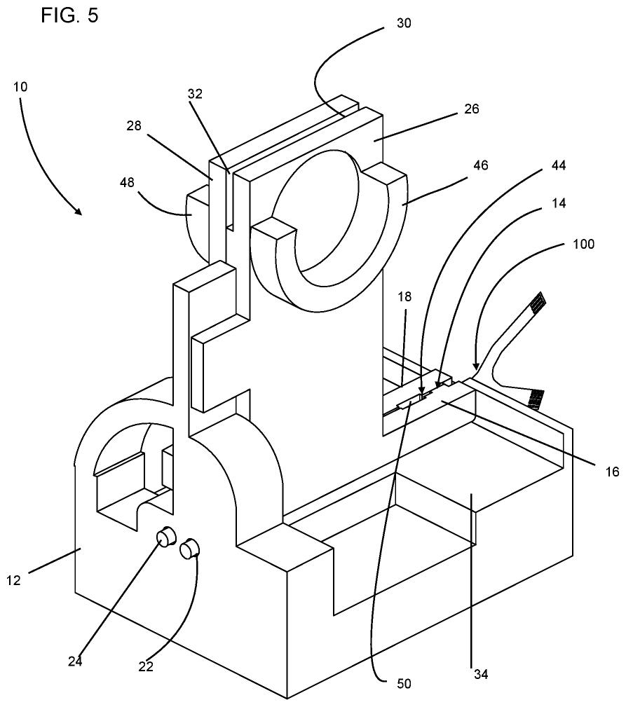

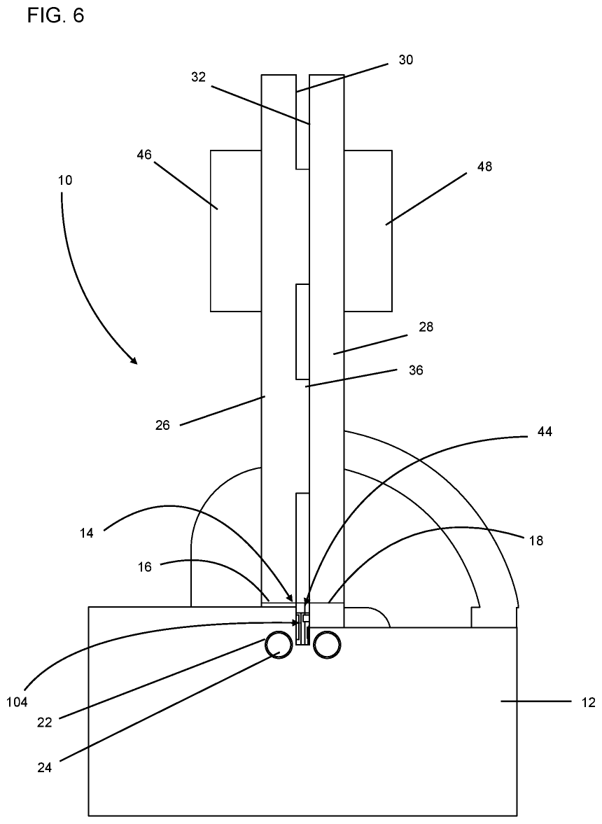

As illustrated in FIG. 1, the tool 10 includes a base 12 and a folding platform 14. The folding platform 14 is comprised of a first portion 16 and a second portion 18. Portions 16 and 18 are positioned relative to the base 12 to define a gap or space 20 between the portions 16 and 18. The base 12 defines two pivot channels 22 through which a pivot member or axle 24 is passed. Each portion 16 and 18 of the folding platform 14 is rotatably or pivotally disposed about one of the axles 24 so that each portion 16 and 18 is capable of pivoting about an axle 24. The gap 20 has a width sufficient to allow the portions 16 and 18 to pivot relative each other and to the base 12 without interference. By this arrangement, the portions 16 and 18 of the folding platform 14 are able to pivotally transition from an open position shown in FIG. 1 to a closed position such as is shown in FIGS. 4-6.

Transition of the folding platform 14 between the open position and the closed position may be accomplished by any of a variety of mechanisms. For example in some embodiments the tool 10 includes one or more electric motors, servos, or similar devices that are in mechanical communication with the portions 16 and 18 of the folding platform 14 and when activated cause portions 16 and 18 to actuate between the open position and the closed position when desired. In other embodiments, the folding platform 14 may be manually actuated by an individual technician (not shown).

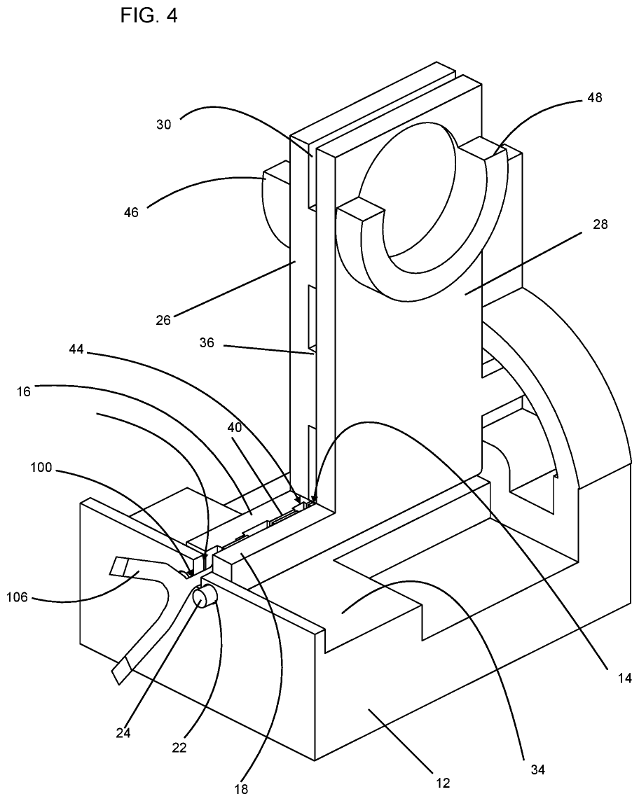

In such a manually actuatable embodiment, such as in the embodiment shown in the present figures, first portion 16 of the folding platform 14 includes a first actuation arm 26 and the second portion 18 includes a second actuation arm 28. The arms are sized and configured to allow a person (not shown) to grip and move the arms 26 and 28 by hand between the open position shown in FIGS. 1 and 3, and the closed position shown in FIGS. 4-6 (i.e., manually actuate the arms), thereby causing the folding platform 14 to be actuated at the same time the arms 26 and 28 are actuated.

First portion 16 and second portion 18 of the folding platform 14 (as well as arms 26 and 28) have an interior face 30 and 32 respectively. In the open position shown in FIG. 1, the interior face 30 of the first portion 16 and the interior face 32 of the second portion 18 are in alignment along a common plane. In the open position each interior face 30 and 32 is substantially parallel with a top surface 34 of the base 12. When the portions 16 and 18 are in the closed position shown in FIGS. 4-6, the interior face 30 of the first portion 16 and the interior face 32 of the second portion 18 are positioned in substantially parallel, spaced apart, alignment with one another but are both substantially perpendicular to the top surface 34 of the base 12.

Note: The term “substantially” as used above is intended to reflect the inexact nature of machine tolerances and normal irregularities that occur when many types of apparatuses are assembled and/or used. For example, in regard to terms such a “substantially parallel” or “substantially perpendicular” the described elements may be in fact parallel or perpendicular to one another, as the case may be, but the elements may also have variabilities in their structure or assembly resulting in a relationship that is other than perfectly parallel or perpendicular but remains substantially so.

Actuation of the arms 26 and 28, and transition of the folding platform 14 portions 16 and 18 from the open position to the closed position is achieved by pivotally moving (relative to the respective axles 24) each the arms 26 and 28 from their nominally at rest position upon the top surface 34 of the base 12 around opposite 90 degree arcs of movement until at least some portions of the interior faces 30 and 32 of the arms 26 and 28 come into contact with one another in the manner shown in FIGS. 4-6.

To ensure that the portions 16 and 18 of the folding platform 14 are only folded to the extent necessary or desired, in some embodiments, one or both of the arms 26 and 28 are provided with protrusions or stops 36 which protrude vertically out from the surrounding interior face 30 or 32 such that they will engage the opposing arm's interior face when the arms 26 and 28 are brought into the closed position. The, position, size and configuration such a stop or stops 36 may be varied to allow the portions 16 and 18 of the folding platform 14 to be held closer or further apart as desired when in the closed position.

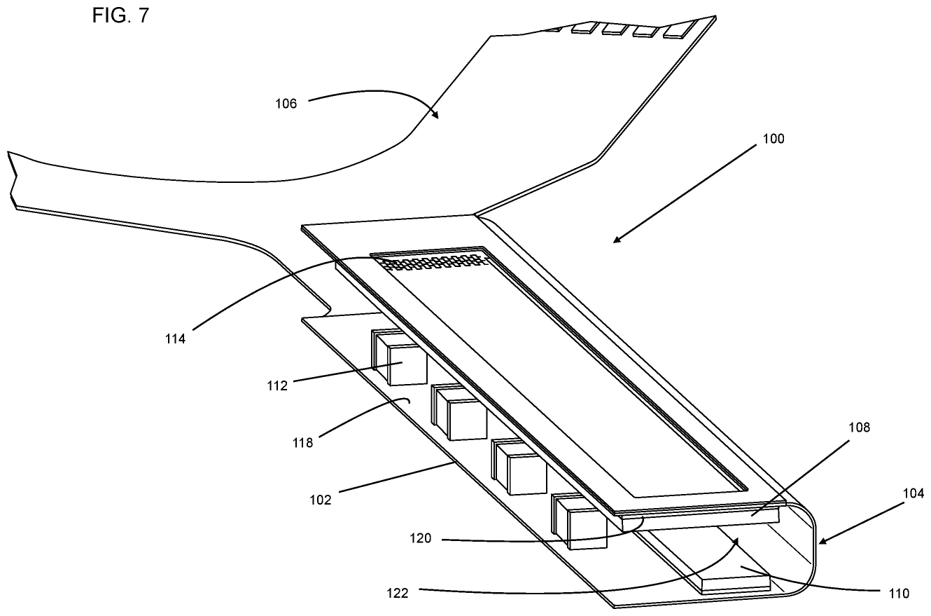

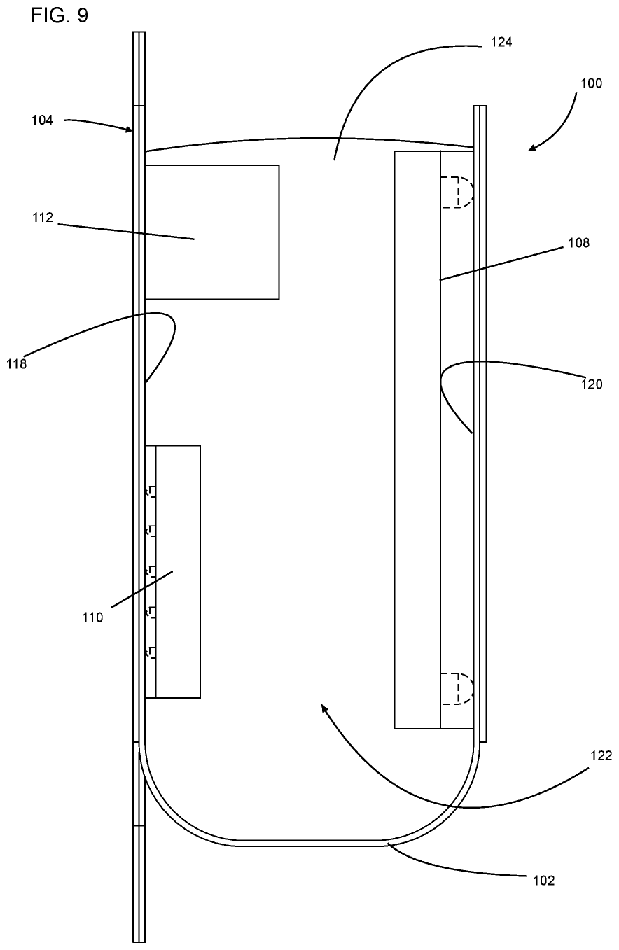

As discussed above, a primary function of the tool 10 is to fold a US transducer assembly 100 from it flattened or manufactured state, an example of which shown is shown in FIG. 2, into a folded configuration whereby the tool 10 imposes upon the assembly a taco-like shape such as is illustrated in FIGS. 7 and 9.

US transducer assemblies are complex electronic components utilized in EBUS devices. The exemplary embodiment of the US transducer assembly 100 shown in FIGS. 2-9 includes a flexible substrate 102 that is divided into two regions or pads, namely an operational pad 104 and a testing pad 106; the testing pad 106 is used to electronically test the operational characteristics of the components mounted to the operational pad 104 and may be removed from the operational pad after testing has occurred.

The components mounted to the operational pad 104 include piezoelectric micromachined ultrasonic transducer (PMUT) element (shown in FIG. 2 as a single array) 108, one or more application specific integrated circuits (ASICs) 110, and one or more capacitors 112 all mounted to the same side of the flexible substrate 102. Each of the PMUT 108, ASICs 110, and capacitors 112 are electrically connected to contacts (not visible, but will be understood by those of ordinary skill in the art to include electrical contacts or traces between the aforementioned components of the assembly) on the same side of the flexible substrate 102.

The flexible substrate may include multiple traces electrically connecting each ASIC 110 to multiple different elements of the PMUT 108. For example, in at least one embodiment, the US transducer assembly 100 may have four ASICs 110 and sixteen electrical traces plus a ground trace connected to each ASIC 110, such that each ASIC 110 controls sixteen elements 114 on the PMUT 108 and the PMUT 108 has a total of sixty-four individually controllable elements 114 (example elements 114 are visible in FIG. 7).

In order to fold the US transducer assembly 100 from its flattened, pre-folded state shown in FIG. 2 into the taco-like shape shown in FIG. 7, the operational pad 104 of the assembly 100 is first placed on the folding platform 14 in the manner shown in FIG. 3. In at least some embodiments, the body 12 of the tool 10, defines a notch 38 (shown in FIG. 1) through which the testing pad 106 may extend outward from the tool 10 when the operational pad 104 is properly positioned on the folding platform 14.

In some embodiments, each portion 16 and 18 of the folding platform 14 includes one or more raised retaining tabs 40 positioned about the external perimeter 42 of each portion 16 and 18 of folding platform 14. A given retaining tab 40 partially overlaps the respective interior face 30 or 32 the portion 16 or 18 of which they are a part.

Functionally, this overlap between the interior face 30 or 32 and a retaining tab 40 forms a space into which an edge 116 (visible in FIG. 2) of the flexible substrate 102 of the US transducer assembly 100 is engaged when the operational pad 104 is positioned on to the folding platform 14. The retaining tabs 40 ensure that during the folding process, the operational pad 104 remains properly seated within the confines of the folding platform 14.

Once the operational pad 104 is positioned on to the folding platform 14 in the manner shown in FIG. 3, the portions 16 and 18 of the folding platform 14 are actuated from their open position to their closed position (shown in FIGS. 4-6) by physical manipulation of the arms 26 and 28 or by other actuation mechanisms in the manner previously described. As the portions 16 and 18 of the folding platform converge as a result of their respective pivoting about axles 24, the flexible substrate 102 of the US transducer assembly 100 will be gradually folded into the desired taco-like shape, shown in FIG. 7, as a consequence.

As previously mentioned, when the portions 16 and 18 of the folding platform 14 are in the open position, such as is shown in FIGS. 1 and 3, the respective faces 30 and 32 of portions 16 and 18 are in substantially parallel alignment along a common plane. When the portions 16 and 18 are pivoted into the the closed position, illustrated in FIGS. 4-6, the aforementioned common plane of the faces 30 and 32 transitions into folding channel 44, which contains the now folded operational pad 104 of the US transducer assembly 100 (visible in FIG. 6, and depicted removed from the confines of the tool 10, in FIG. 7). Though the flexible substrate 102 of the assembly is indeed sufficiently flexible to allow the operational pad 104 to be folded in the manner shown and described, in the embodiment shown, the material of the substrate 102 is not capable of maintaining the operational pad 104 in the folded state without the use of an adhesive or some other mechanism configured to hold the opposing sides 118 and 120 (shown in FIGS. 7 and 9) of the flexible (now folded) substrate 102 together.

In some embodiments, the channel 44 is substantially U-shaped.

In some embodiments the flexible substrate 102 is composed of a shape memory material that is programed to maintain the flexible substrate 102 in the folded state once the operational pad 104 is placed in that position via the tool 10.

In those embodiments where an adhesive is required to maintain the operational pad 104 of the US transducer assembly 100 in its folded state, it may be necessary to hold or otherwise maintain the assembly 100 in folded state while adhesive is appropriately applied. In some embodiments, the arms 26 and 28 may simply be held together by the tool's operator (not shown). In at least one embodiment, the tool 10 provided with a securement mechanism such a latch, lock or similar mechanism that retains the portions 16 and 18 of the folding platform 14 in the closed position, despite any biasing opposition that the folded assembly 100 might be exerting.

In the present embodiment shown in FIGS. 4-6, the securement mechanism is in the form of a first magnet 46 and a second magnet 48. The first magnet 46 is positioned along the first portion 16 of the folding platform 14. In the embodiment shown, the first magnet 46 is retained on the first arm 26. Likewise, the second magnet 48 is positioned along the second portion 18 of the folding platform 14. In the embodiment shown, the second magnet 48 is retained on the second arm 28. The first magnet 46 and the second magnet 48 are positioned on their respective portions 16 and 18 such that their opposing poles are in closest proximity to one another when the folding platform 14 is in the closed position. As a result, when the portions 16 and 18 are in the closed position the opposing polarities of the magnets 46 and 48 will hold the portions 16 and 18 in the closed position for as long as desired without the need for other types of manual retention force being applied. The closed position may be maintained until an opposing force is applied to the arms 26 and 28 to overcome the magnetic attraction of the magnets 46 and 48.

In at least one embodiment, the first portion 16 and the second portion 18 of the folding platform 14 are magnetically attracted to each other. In at least one embodiment, at least a portion of the first arm 26 and at least a portion of the second arm 28 are magnetically attracted to each other.

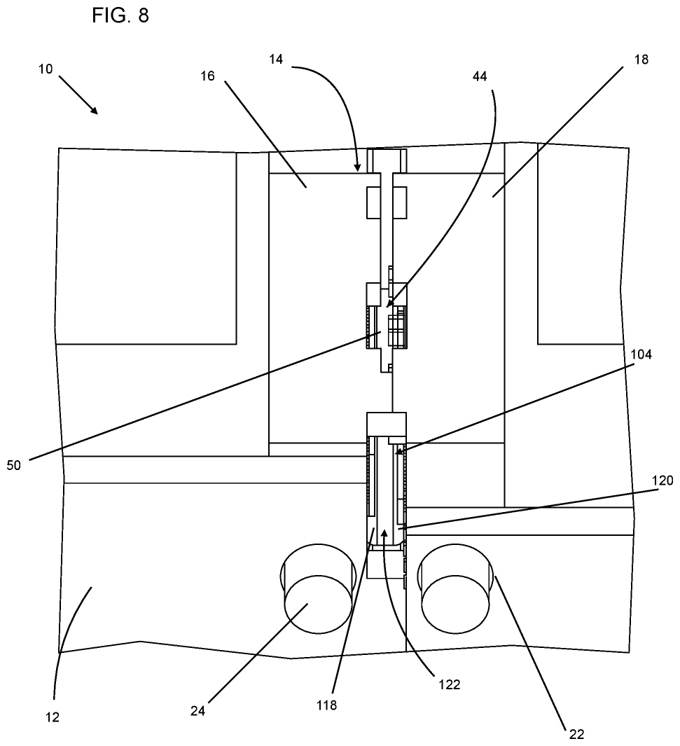

As mentioned above, the operational pad 104 in the folded state has opposing sides 118 and 120, which define a fold or interior 122 of the taco-like shape that the operation pad 104 takes on in the folded state (best shown in FIG. 7). In those embodiments where an adhesive is needed to secure the sides 118 and 120 together, it is necessary to inject or otherwise apply an adhesive this interior 122 space. To facilitate adhesive injection into the interior 122 of the operational pad 104, in some embodiments, an example of which is illustrated in FIG. 8, the folding platform 14 of the tool 10 defines at least one injection port 50 which is in fluid communication with the folding channel 44 and the interior 122 of any operational pad 104 contained therein. Adhesive 124 (shown in FIG. 9) may be injected into the interior 122 of the operational pad 104 through the port(s) 50. The portions 16 and 18 of the folding platform 14 are maintained in the closed position for as long as is necessary to allow the adhesive 124 to dry, cure, or otherwise reach a state sufficient to hold the operational pad 104 in a permeant folded taco-like shape shown in FIG. 9.

In some embodiments the injection port(s) 50 are merely gaps between correspondingly positioned retaining tabs 40 of each portion 16 and 18 of the folding platform 14.

The many features and advantages of the disclosure are apparent from the above description. Numerous modifications and variations will readily occur to those skilled in the art. Since such modifications are possible, the disclosure is not to be limited to the exact construction and operation illustrated and described. Rather, the present disclosure should be limited only by the following claims.

What is claimed is:

1. An medical device folding apparatus comprising:
a base, the base having a top surface, the base defining two pivot channels;
a first axle and a second axle, each axle passing through one of the pivot channels;
a folding platform, the folding platform having a first portion and a second portion, the first portion separated from the second portion by a gap, the first portion pivotally disposed about the first axle and the second portion pivotally disposed about the second axle, the first portion defining a first portion interior face, the second portion defining a second portion interior face;
the folding platform being moveable between an open position and a closed position, in the open position the first portion interior face and the second portion interior face are in substantially parallel alignment with the top surface of the base,
in the closed position the first portion interior face and the second portion interior face are each substantially perpendicular to the top surface of the base, and the first portion interior face and the second portion interior face define a folding channel.

2. The apparatus of claim 1, the base defining a notch in communication with the folding channel.

3. The apparatus of claim 1, wherein the first portion and a second portion each define a perimeter, along the perimeter of each of the first portion and second portion are located at least one engagement tab.

4. The apparatus of claim 1, wherein the first portion includes a first actuation arm and the second portion includes a second actuation arm, at least a portion of the first actuation arm including the first portion interior face, at least a portion of the second actuation arm including the second portion interior face,
at least one protrusion extending vertically from at least one of the first portion interior face and the second portion interior face,
the at least one protrusion constructed and arranged to prevent the first portion interior face and the second portion interior face from coming into direct contact in the closed position.

5. The apparatus of claim 1, wherein the folding channel is substantially U-shaped.

6. The apparatus of claim 1, wherein in the closed position the first portion and the second portion define at least one port in fluid communication with the folding channel.

7. The apparatus of claim 1, wherein the first portion and a second portion each define a perimeter, positioned along the perimeter of each of the first portion and second portion are a plurality of engagement tabs, the plurality of engagement tabs being spaced apart from one another.

8. The apparatus of claim 7, wherein in the closed position each of the plurality of engagement tabs of the first portion are in contact with an opposing engagement tab of the plurality of engagement tabs of the second portion.

9. The apparatus of claim 8, wherein at least one of the spaces between the plurality of engagement tabs defines a port in fluid communication with the folding channel.

10. The apparatus of claim 9, wherein the medical device is a ultrasound (US) transducer assembly.

11. The apparatus of claim 10, wherein the folding platform is constructed and arranged to receive at least a portion of the US transducer assembly in its manufactured state.

12. The apparatus of claim 11, wherein the at least a portion of the US transducer assembly defines and edge, the edge being engaged by the plurality of engagement tabs when the at least a portion of the US transducer assembly is received by the folding platform.

13. The apparatus of claim 12, wherein the at least a portion of the US transducer assembly includes a flexible substrate, in the closed position the flexible substrate of the at least a portion of the US transducer assembly being folded into a taco-like shape.

14. The apparatus of claim 13, wherein the taco-like shape of the flexible substrate defines opposing interior sides, the opposing interior sides defining an interior, the interior in fluid communication with the port.

15. The apparatus of claim 14, further comprising an adhesive, the adhesive being injected into the interior through the port.
